(19) United States
(12) Patent Application Publication
(10) Pub. No.: US 2005/0165462 A1
(43) Pub. Date: Jul. 28, 2005

(54) Light delivery device using conical diffusing system and method of forming same

(76) Inventors: Roland Bays, Ecublens (CH); Laurent Mosimann, Commugny (CH); Alain Woodtli, Saint-Aubin (CH); Wai-Fung Cheong, Los Altos, CA (US)

Correspondence Address: Townsend and Townsend and Crew, Two Embarcadero Center, Eighth Floor, San Francisco, CA, 94111-3834, US

(21) Appl. No.: 10/503,692

(22) PCT Filed: Feb. 5, 2003
(86) PCT No.: PCT/US03/03569

Related U.S. Application Data
(60) Provisional application No. 60/355,736, filed on Feb. 5, 2002.

(51) Int. Cl. A61N 1/00

(52) U.S. Cl. 607/88

(57) ABSTRACT

The present invention provides devices, methods of manufacture, methods of use and kits related to transmitting and diffusing light for delivery to a target site. Techniques are provided which allow accurate control of the illumination profile with a diffuser tip design which is easily produceable, relatively inexpensive and provides countless variations to obtain desired illumination profiles. This is achieved with the use of at least one scattering region having a conical shape. The number of conical scattering regions, the dimensions of such region(s), and the scattering properties of the scattering materials may be selected individually and/or collectively to selectively control the resulting illumination profile. In addition, the conical features allow for other beneficial design features, such as a smaller cross-sectional diameter than is typically achievable with other techniques. The resulting light transmission and diffusion apparatus is operable with a high efficiency, highly predictable illumination profile and ease of use.

### BACKGROUND OF THE INVENTION

1. Field of the Invention

The present invention relates to apparatus, methods of manufacture, and methods of use for transmitting and diffusing light for delivery to a target site to be illuminated, heated, irradiated, or treated by exposure to light. Particularly, the present invention relates to the delivery of light to a body lumen or body cavity for photodynamic therapy of atherosclerosis, malignant or benign tumor tissue, cancerous cells and other medical treatments. Photodynamic Therapy (PDT) is a known method of treating target regions or sites, such as tumors, atheromatous plaques and other tissues, in humans by administering a photosensitizing substance to a patient and allowing it to concentrate preferentially in the target sites. It has been found that certain abnormal growths, such as certain cancerous tissue and atheromatous plaque, have an affinity for these photosensitizing agents. Photosensitizing agents are compounds that, when exposed to light, or light of a particular wavelength or wavelengths, create O₂ radicals which react with the target cells. Examples of such agents include texaphyrins, hematoporphyrin, chlorins, and purpurins. In the case of living cells, such as cancer tumors, an appropriate photosensitizing agent is used to create the O₂ radicals which kill the target cells. In other situations, such as when it is desired to destroy atheromatous plaque tissue, an appropriate photosensitizing agent is activated to destroy the plaque by lysis (breaking up) of such plaque. Mechanisms other than lysis, e.g. cell apoptosis, may also be involved.

Photoactivation of the photosensitizer is achieved by locally delivering light to the target region, preferably in a manner which achieves an optimum “dose” and emission configuration which is consistent with the volume and geometry of the target tissue. This may be accomplished through the use of light delivery systems which utilize optical fibers. For example, for tubular body areas and lumens, such as a bronchus, esophagus or blood vessel, it is common to use a fiber optic diffuser which distributes the light in a cylindrical pattern. Thus, for PDT treatment of esophageal cancer, an optical fiber may be equipped with an apparatus at its tip which disperses light propagating along the fiber in a uniform cylindrical pattern with respect to the central axis of the optical fiber. Uniformity is usually desired to ensure delivering a known and optimum dose.

A number of diffuser tip designs have been developed to produce a controlled and generally uniform profile of illumination. One approach involves modifying a distal segment of the waveguide, typically an optical fiber. Such modifications include etching the fiber cladding or creating fiber gratings within the fiber core. Another approach involves launching light from the tip of a waveguide into a diffuser tip containing scattering medium, wherein the light is launched in a primarily axial direction and is distributed radially outward by the optical scattering medium. Often it is desired that the scattering medium have a uniform scattering property. Thus, many designs aim to uniformly embed scattering particles throughout an optically clear medium. In addition, a mirror is often placed at the distal end of such a diffuser tip to reflect light which has not been sufficiently diffused during its first pass through the scattering medium.

Although the scattering medium approach typically produces more robust and highly flexible diffuser tips, a number of difficulties arise with this approach. First, uniform light distribution is difficult to achieve with current designs when the diffuser tip is long and narrow, particularly if the tip is desired to be flexible. Second, the illumination profile may only be controlled by one parameter for a given tip length, the diffusion property of the scattering medium. This makes it difficult to obtain a uniform “top hat” illumination profile with sharply demarcated edges. Third, if a high quantity of light is reaches the mirror, the mirror absorbs some of the light and can consequently warm up. High quality mirrors with dielectric coatings and no edge imperfections are needed to reduce such warming. And fourth, fixing a mirror at the end of a flexible and soft scattering medium to provide controlled reflection properties is often difficult to achieve, particularly in small diameter diffuser tips (e.g. less than 0.18 inches or 450 μm). Such small diameter tips may be used in treating obstructions in the coronary arteries and may require a diffuser tip of approximately 0.14 inches (350 μm) or less.

To overcome some of these difficulties, diffuser tip designs have utilized a light scattering medium having continuously increasing optical scattering power in a direction parallel to the central axis of the tip in attempts to maintain uniform circumferential scattering power. The increasing scattering power is obtained by continuous variation of the concentration of scattering particles embedded in the core medium along the length of the tip. However, there are practical difficulties in obtaining both the uniform circumferential scattering power and the continuously increasing scattering power along the length of the tip. In an effort to overcome these difficulties, discontinuous sections of scattering medium have been used along the length of the tip, each section having an increased scattering power. With this design, circumferentially uniform scattering power is still difficult to obtain since the discontinuous sections do not provide smooth transitions. In addition, if this design is used without a reflecting mirror at the end of the diffusing medium, a large number of discrete sections of scattering medium are required.

For these reasons, it would be desirable to provide a light transmission and diffusion apparatus which overcome at least some of the shortcomings discussed above. In particular, it would be desirable to provide such an apparatus having a diffuser tip which delivers a uniform illumination profile by means of a design which is practically achievable, manufacturable, and controllable. It would be further desirable to provide such a diffuser tip design which is easily adapted to provide other desired illumination profiles. In addition, such designs should be adaptable to various dimensional parameters, particularly small outer diameter for access to small vessels, such as coronary arteries. This may include the elimination of a reflective mirror fixed at the end of the diffuser tip and/or the addition of a guidewire lumen. Further, it would be desirable to provide methods of manufacture, methods of use and kits related to such an apparatus.

2. Description of the Background Art

Anderson (U.S. Pat. No. 5,814,041) describes an illuminator comprising a differential optical radiator having two regions, each having different reflectivities and therefore transmissivities, and a laser fiber disposed within the differential optical radiator. The laser fiber includes a diffusively reflective coating. The radiator is described to produce a substantially uniform pattern of illumination from said first and second regions.

Hashimoto (EP 673627) and Hashimoto et al. (U.S. Pat. No. 6,152,951) describe a cancer therapeutic instrument having an optical fiber emitting from its tip activation light toward scatter member.

Sinofsky (WO 96/07451) describes a diffusive tip apparatus for use with an optical fiber for diffusion of radiation propagating through the fiber. Related U.S. Pat. No. 5,632,767 describes an apparatus having a tip assembly for directing radiation outward wherein each tip assembly is arranged in a loop configuration to form a loop diffuser. U.S. Pat. No. 5,637,877 describes an apparatus for sterilizing an endoscopic instrument lumen. U.S. Pat. No. 5,643,253 describes an apparatus having a sheath surrounding an optical fiber having a fluted region which is capable of expanding upon penetration of the optical fiber into biological tissue. And U.S. Pat. No. 5,908,415 describes an apparatus having a tip assembly which relies on a reflective end surface to retransmit some of the light back through the scattering medium providing an axial distribution over the length of the scatterer tube when combined with the initially scattered light.

Esch (U.S. Pat. No. 5,754,717) claims a device for diffusing light having a tip composed of a material characterized by low light absorption to avoid producing a hot tip.

Mersch (U.S. Pat. No. 5,693,049) describes an apparatus comprising a tubular catheter and an optical coupler for coupling light radiation to the catheter, which diffuses the light radiation outwardly therefrom within a blood vessel to irradiate blood flowing through the blood vessel.

Overholt (WO 9743966) describes a device that is able to irradiate a segment of tissue that is 4 cm or longer. Overholt et al. (U.S. Pat. No. 6,146,409) describes a balloon catheter having a treatment window, that is at least 4 cm in length, and a diffuser that extends beyond the distal and proximal ends of the treatment window. The window and diffuser function or cooperate together to provide uniform light in a single effective dose.

Narciso (U.S. Pat. No. 5,169,395) describes a guidewire-compatible intraluminal catheter for delivering light energy in a uniform cylindrical pattern.

Fuller (U.S. Pat. No. 5,807,390) describes a probe having a tip consisting essentially of light propagating material having inclusions distributed therein and generally throughout; the light propagating material being a light propagating inorganic compound, wherein the inclusions include microscopic voids having dimensions substantially smaller than the wavelength of the light energy.

Doiron (U.S. Pat. No. 5,269,777) describes a diffuser tip comprising an optical fiber and a terminus comprising a second core consisting of a substantially transparent elastomer which is concentrically surrounded by a layer having light-scattering centers embedded therein.

Willing (DE 4,329,914) describes a linear optical waveguide having cut-out elements arranged at surface and/or in volume of light waveguide which allow part of rays in waveguide to emerge from waveguide.

Rowland (WO 9000914) describes a device for illuminating a flexible stricture in a tube, comprising an illuminator body provided with a transparent window and adapted to be passed down the tube and a light source in the illuminator body, for illuminating the window the illuminator body being so adapted that a known quantity of light can be directed onto the stricture.

Kakarni (U.S. Pat. No. 5,078,711) describes a laser irradiation device having a changeable irradiation angle of laser light.

Additional patents relating to light delivery devices and methods include U.S. Pat. Nos. 5,903,695; 5,871,521; 5,861,020; 5,851,225; 5,836,938; 5,833,682; 5,797,868; 5,766,222; 5,728,092; 5,723,937; 5,718,666; 5,709,653; 5,700,243; 5,695,583; 5,695,482; 5,671,314; 5,645,562; 5,620,438; 5,607,419; 5,588,952; 5,542,017; 5,536,265; 5,534,000; 5,530,780; 5,527,308; 5,520,681; 5,514,669; 5,496,308; 5,479,543; 5,478,339; 5,456,661; 5,454,794; 5,454,782; 5,453,448; 5,441,497; 5,432,876; 5,431,647; 5,429,635; 5,401,270; 5,373,571; 5,372,756; 5,363,458; 5,354,293; 5,348,552; 5,344,419; 5,337,381; 5,334,206; 5,330,465; 5,312,392; 5,303,324; 5,292,320; 5,267,995; 5,253,312; 5,248,311; 5,219,346; 5,217,456; 5,209,748; 5,207,669; 5,196,005; 5,193,526; 5,190,538; 5,190,535; 5,151,096; 5,139,495; 5,129,897; 5,119,461; 5,074,632; 5,073,402; 5,059,191; 5,054,867; 5,042,980; 5,032,123; 4,995,691; 4,989,933; 4,986,628; 4,927,231; 4,889,129; 4,878,725; 4,878,492; 4,860,743; 4,848,323; 4,842,390; 4,840,174; 4,782,818; 4,763,984; 4,736,745; 4,733,929; 4,732,442; 4,693,556; 4,693,244, 4,676,231; 4,660,925; 4,612,938; 4,528,617; 4,471,412; 4,466,697; 4,422,719; 4,420,796; 4,336,809; 4,248,214; 4,195,907; Re 34544.

Additional foreign patents and applications relating to light delivery devices and methods include WO 9923041; WO 9911323; WO 9911322; WO 9904857; WO 9848690; WO 9811462; WO 9743965; WO 9629943; WO 9607451; WO 9509574; WO 9325155; WO 9321841; WO 9321840; WO 9318715; WO 9004363; WO 9002353; EP 772062; EP 732086; EP 732085; EP 732079; EP 292621; EP 394446; EP 391558; EP 433464; EP 377549; EP 561903; EP 6022051; DE 2853528 DE 19507901; GB 2323284; GB 2154761; JP 5011852; AU-A-64782/90.

### SUMMARY OF THE INVENTION

The present invention provides devices, methods of manufacture, methods of use and kits related to transmitting and diffusing light for delivery to a target site. Such delivery of light is useful in Photodynamic Therapy (PDT), a method of treating target sites in the human body, such as tumors, atheromatous plaques and other disease tissues. Typically, intraluminal, intracavity, or interstitial PDT is performed with the use of a light guide having a diffuser tip located at its distal end. Light traveling axially through the light guide is then radially dispersed through the diffuser tip to treat the target site. The present invention achieves accurate control of the illumination profile with an improved diffuser tip design which is easily produceable, relatively inexpensive and provides countless variations to obtain desired illumination profiles. The diffuser comprises at least one scattering region having a conical shape. The number of conical scattering regions, the dimensions of such region(s), and the scattering properties of the scattering materials, among other features, may be selected individually and/or collectively to selectively control the resulting illumination profile. Uniform illumination profiles which are typically difficult to accurately produce may be more easily achievable with the techniques of the present invention. Further, alternative profiles may also be achieved by altering design choices in a controlled manner. In addition, the conical features allow for other beneficial design features, such as a smaller cross-sectional diameter than is typically achievable with other techniques. The resulting light transmission and diffusion apparatus is operable with a high efficiency, highly predictable illumination profile and ease of use.

In a first aspect of the present invention, a light transmission and diffusion apparatus is provided for use in delivering light to a target site, such as for treatment or diagnostic purposes. The apparatus comprises a light guide which transmits light from a light source to a diffuser tip. The diffuser tip diffuses the received light in a controlled pattern, described as an illumination profile. Delivery of the diffused light to the target site provides specific treatment depending on the profile, duration and intensity of the light. Thus, various embodiments of the diffuser tip provide different illumination profiles and therefore different treatment and/or diagnostic options.

In a first embodiment, the light guide has a proximal end and a distal end, the proximal end adapted for coupling to a light source and a distal end having a light transmitting end portion. In addition, the diffuser tip has a proximal end, enclosing the light transmitting end portion, and a distal end. The tip comprises a number of regions, each region having a specific shape, dimension and material to create an optical effect. Each tip comprises at least two regions. The first region may be of any shape and may comprise any suitable medium, such as a transparent material or a light scattering medium. The second region has a conical shape and is comprised of a light scattering medium or a partially light scattering and partially light absorbing medium. Although the second region may be distal to the first region, the second region is proximal to the distal end of the diffuser tip. In other words, the distal end of the diffuser tip may have any shape, square, round, conical or other, but the second region is separate from and proximal to this distal end. Thus, if the diffuser tip has a conically shaped distal end having an apex, the diffuser tip will also have a conically shaped second region which is separate from this having its own apex. Such an example would be a diffuser tip having a conically shaped second region, with its apex facing the light transmitting end portion, and a conically shaped distal end facing distally.

By providing a diffuser tip comprising a conically shaped region having light scattering properties, light entering the diffuser tip is diffused and redirected in a unique manner which affords a number of advantages. To begin, since the conical region varies in dimension from its apex to its base, light will enter or exit the conical region in a gradual pattern. This affords a smoother transition between regions having different scattering powers. In addition, the conical shape provides an effective “overlap” or nesting of regions having different scattering properties. Thus, light scattered radially outward from the axial center of the diffuser tip may be directed through more than one scattering material adding higher levels of scattering control. By adding more cones, and thus more layers, the scattering effect may be more highly defined and manipulated. Likewise, by varying the scattering materials in the cones, the scattering effect may be additionally manipulated. Thus, a number of illumination profiles may be created depending on the type, number, nesting and arrangement of the conical scattering regions.

In preferred embodiments, the conical second region is oriented so that its apex is directed toward the light-transmitting end portion. Thus, the conical region increases in width toward the distal end of the diffuser tip and therefore its scattering power naturally increases monotonically. This design provides a high efficiency or ratio between the light power emitted from diffuser tip and the light power coupled to the proximal end of the light guide. Most light is propagated through the tip and a minimum quantity is emitted back to the light guide by backscattering induced by the cone. Simulations and experiments have shown that introduction of a conical region in this orientation does not affect the light distribution proximal to the apex and only causes local effects in the area of the cone. It may be appreciated that in other embodiments the conical second region is oriented in a direction other than toward the light-transmitting end portion. In this case, least some of the above described advantages are still afforded.

As mentioned, additional regions, such as a third region, fourth region, fifth region, sixth region, seventh region, eighth region, ninth region, tenth region or more, can be included in the diffuser tip. Such additional regions may have any shape and may be comprised of any medium, including transparent material, light absorbing, light scattering mediums and mediums which partially scatter and partially absorb. Although more than one region in a diffuser tip may be comprised of the same material having the same concentration of scattering particles, and therefore the same scattering power, each such region is separated by a region having a different scattering power. In some embodiments, the additional regions have a conical shape and are oriented so that each apex is directed toward the light-transmitting end portion. Typically, these conical regions have bases which are aligned and apexes which are disposed at different distances from the bases though each pointing toward the light-transmitting end portion.

Also, in some embodiments, each region has an increasing scattering power in the direction of the distal end. This may be achieved by the incorporation of higher and higher concentrations of scattering particles in each region toward the distal end. This may culminate in the distal end being opaque wherein any remaining unscattered light will not pass through the distal end. This design may eliminate the need for a mirror placed at the distal end of the diffuser tip. Typically such mirrors reflect light from the distal end back toward the light transmitting end portion. However, this increases inefficiency, can lead to heating of the mirror and is difficult to manufacture, particularly with diffuser tips having small cross-sectional diameters.

Thus, as described above, the diffuser tip may be comprised any number of regions wherein at least one has a conical shape with light scattering properties. Such regions may be arranged in any orientation and may be comprised of any light scattering, transparent or other material. Other materials may include particles providing optical properties other than or in addition to scattering, such as light absorbing particles, fluorescent particles, or magnetic resonance imaging (MRI)-detectable particles. Such optical properties may allow the region to be used for detectors, sensors or MRI-guided placement of the diffuser tip, in addition to light therapy treatment. This may reduce the need for fluorscopy in placement of the diffuser tip. In a preferred embodiment, the diffuser tip is comprised of a first region disposed adjacent to the light transmitting end portion and a number of additional regions, each conical in shape and oriented so that their apexes are directed toward the end portion.

In any case, the apparatus provides an illumination profile resulting from the design choices of the regions within the diffuser tip. In one embodiment, the regions are positioned and their light scattering mediums and concentrations of scattering particles are chosen such that the diffuser tip produces a substantially uniform pattern of light emission. Alternatively, the regions may be shaped, arranged and comprised of specific mediums which will provide different illumination profiles. For example, the light intensity may be increased near the proximal and distal ends relative to a plateau of lesser intensity therebetween. This profile may compensate for effects near the ends of the diffuser tip which would otherwise provide diminished light intensity at the target tissue. Thus, any desired illumination profile may be achieved by altering the shape, size, arrangement, orientation, choice of scattering medium, concentration of scattering particles and other variables related to the regions within the diffuser tip.

In second aspect of the present invention, the light transmission and diffusion apparatus may include additional optional features. First, the apparatus may include markings which are used for visualization purposes during treatment. Marking may include radiopaque markings, bands or coatings which are visible under fluoroscopic conditions. Typically such markings are positioned close to a region having light scattering properties, such as near one end, the other end or both ends of the region. Alternatively, one or more regions may be comprised of a material which provides radiopacity, such as barium. Second, the apparatus may include a guidewire lumen. Typically, the guidewire lumen is disposed along an axis which is offset from the central axis of the apparatus. For example, the guidewire lumen may be positioned outside of the scattering regions of the diffuser tip, possibly along the outside edge of the apparatus. The guidewire lumen may extend from the distal end of the diffuser tip to any location along the apparatus. In any case, when a guidewire lumen is present, a guidewire will be positioned within the guidewire tubing during delivery of light therapy to the target site. In the area of the diffuser tip, the guidewire tubing is comprised of a transparent material that allows passage of visible light so that the guidewire tubing will not interfere with the delivery of light to the target region.

In a third aspect of the present invention, the light transmission and diffision apparatus may be adapted to be introduced through other devices or instruments. For example, the diffuser tip may be adapted to be insertable within a lumen in a catheter. Such a catheter may be a transit catheter or a balloon catheter. Such procedures will be discussed in more detail related to methods of the present invention.

According to the methods of manufacturing the present invention, the light transmission and diffusion apparatus is processed by a number of steps. One step involves providing a segment of external tubing having a proximal end, a distal end and a lumen therethrough having a center axis. In addition, the segment has a light guide having an light transmitting end portion disposed within the tubing so that there is a luminal space between the end portion and the distal end. It is primarily within this luminal space that the above described regions will reside. Thus, another step involves creating a first region by injecting a first medium into the luminal space from the distal end. And, still another step involves creating a second region by injecting a second medium into the distal end wherein the second region has a conical shape. When the step of creating the second region is performed after the step of creating the first region, the second medium essentially pushes the first medium through the tubing toward the light transmitting end portion. Due to the flow dynamics in a tube, the velocity of the flowing material reaches a maximum near the central axis of the lumen. Since the second medium is traveling at a higher velocity near the central axis, the second region forms a conical shape wherein the apex is directed toward the end portion. This process can be repeated by adding a third region by injecting a third medium into the distal end wherein the third region has a conical shape. Similarly, additional regions may be added by similar injection steps. The length and shape of the cones may be controlled by the method of injection, including speed of injection, angle and position of the injection tube and a variety of other variables. In addition, it may be appreciated that regions may be non-conical shaped by using other methods of injection. Further, conical regions, wherein the apex is not directed toward the end portion may be produced by injecting material through the tubing wall toward the distal end or by producing the diffuser tip itself and then connecting the diffuser tip to the light guide.

It may be appreciated that the light guide may be comprised of an optical fiber. In this case, the optical fiber may be comprised of a cylindrical core, a cladding layer surrounding the cylindrical core, and a protective buffer encasing the cladded fiber. In this case, a length of the buffer will be removed from the light transmitting end portion, to reveal a length of the cylindrical cladded core.

According to the methods of the present invention, the apparatus of the present invention may be used for performing photodynamic therapy at a target site within a body, such as interstitially or within a body lumen or cavity. Photodynamic therapy involves the use of photosensitive compounds which are introduced to the target site prior to light delivery. Typically the photosensitizing agents are administered to the patient and allowed to concentrate preferentially in the target sites which have an affinity for the agents. The light transmission and diffusion apparatus of the present invention is then introduced to the target site and light radiation is coupled to the apparatus so that light transmitted and received by the diffuser tip is delivered to the target site. Such introduction may be accomplished in a number of ways. When the body lumen is a blood vessel, the introducing step may further comprise advancing the distal end of the apparatus through the vasculature from a location remote from the target site. This location may be accessed percutaneously, such as using needle access as in the Seldinger technique, or by performing a surgical cut down procedure or minimally invasive procedure.

The apparatus may also be introduced to the target site through another device or apparatus. For example, a catheter having a lumen therethrough may first be positioned within the target site. The light transmitting and diffusing apparatus may then be introduced through the catheter lumen so that the diffuser tip is also positioned within the target. The apparatus may then deliver light to the target site wherein the light is dispersed through the walls of the catheter. Alternatively, the catheter may be retracted while the apparatus remains in place. In another example, a balloon catheter having a balloon mounted on its distal end may be positioned within the target site. In this example, target site may comprise an atheromatous stenosis and the balloon catheter is used to perform an angioplasty procedure. While the balloon is inflated, the apparatus may be introduced through the balloon catheter so that the diffuser tip is also positioned within the target site. The apparatus may then deliver light to the target site wherein the light is transmitted through the balloon. Alternatively, the balloon may be deflated and the balloon catheter may be retracted while the apparatus remains in place.

The methods and apparatuses of the present invention may be provided in one or more kits for such use. The kits may comprise a light transmission and diffusion apparatus and instructions for use. Optionally, such kits may further include any of the other system components described in relation to the present invention and any other materials or items relevant to the present invention.

Other features and advantages of the invention will appear from the following description in which the preferred embodiments have been set forth in detail in conjunction with the company drawings.

### BRIEF DESCRIPTION OF THE DRAWINGS

FIG. 1 is a perspective view illustration which depicts an embodiment of the light transmission diffusion apparatus of the present invention.

FIGS. 2-4 provide side views of various embodiments of the diffuser tip of the present invention.

FIG. 5 illustrates the diffusion of light rays delivered from the light transmission diffusion apparatus.

FIG. 5A illustrates light scattered from a conical region.

FIGS. 6A-6B are graphical representations of possible scattered light illumination profiles deliverable by the apparatus.

FIGS. 7A-7C illustrate example distal end shapes of the diffuser tip.

FIGS. 8-10 provide side views of additional embodiments of the diffuser tip of the present invention.

FIGS. 11A-11E illustrate how the present invention may be processed in manufacturing.

FIG. 12 depicts an embodiment of the apparatus including a guidewire lumen.

FIG. 13 illustrates a cross-sectional view of a target site within body lumen.

FIG. 14 illustrates methods of delivering light to a target site with the use of the apparatus of the present invention.

FIGS. 15A-15B depict steps of including the use of a catheter in the methods of introducing the apparatus of the present invention.

FIGS. 16A-16B depict steps of including a balloon catheter in the methods of introducing the apparatus of the present invention.

FIG. 17 illustrates a kit constructed in accordance with the principles of the present invention.

### DETAILED DESCRIPTION OF THE INVENTION

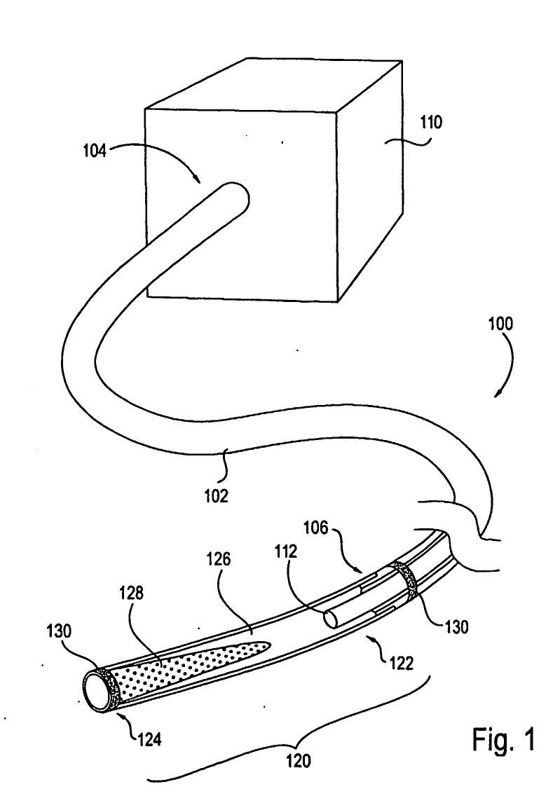

The present invention provides for the transmission and diffusion of light to a target site. This is achieved with the use of a light transmission and diffusion apparatus 100, an embodiment of which is illustrated in FIG. 1. In this embodiment, the apparatus 100 comprises a light guide 102 having a proximal end 104 and a distal end 106, the proximal end 104 adapted for coupling to a light source 110 and the distal end 106 having a light-transmitting end portion 112. In addition, the apparatus 100 comprises a diffuser tip 120 having a proximal end 122 enclosing the end portion 112 and a distal end 124. The tip comprises at least a first region 126 and a second region 128, wherein the second region 128 has a conical shape. Optionally, the apparatus 100 may also include radiopaque markers 130, possibly one located near the proximal end 122 and one near the distal end 124 of the diffuser tip 120 as shown, to aid in visualization during use. Typically, as shown, the apparatus 100 has an elongated, cylindrical shape with a blunt or curved distal end. Such a shape is adapted for use in treating cylindrical target locations, such as body lumens, or in reaching target locations which are accessible by similarly shaped pathways. Alternatively, the apparatus 100 may have other shapes conducive to other purposes. Further, the distal end 124 may have various shapes depending on usage. In general, the apparatus 100 is usually approximately 2-5 meters in total length with an outer diameter of 100 microns to 2 mm, preferably at least 2001 μm. The diffuser tip is typically approximately 1-15 cm in length.

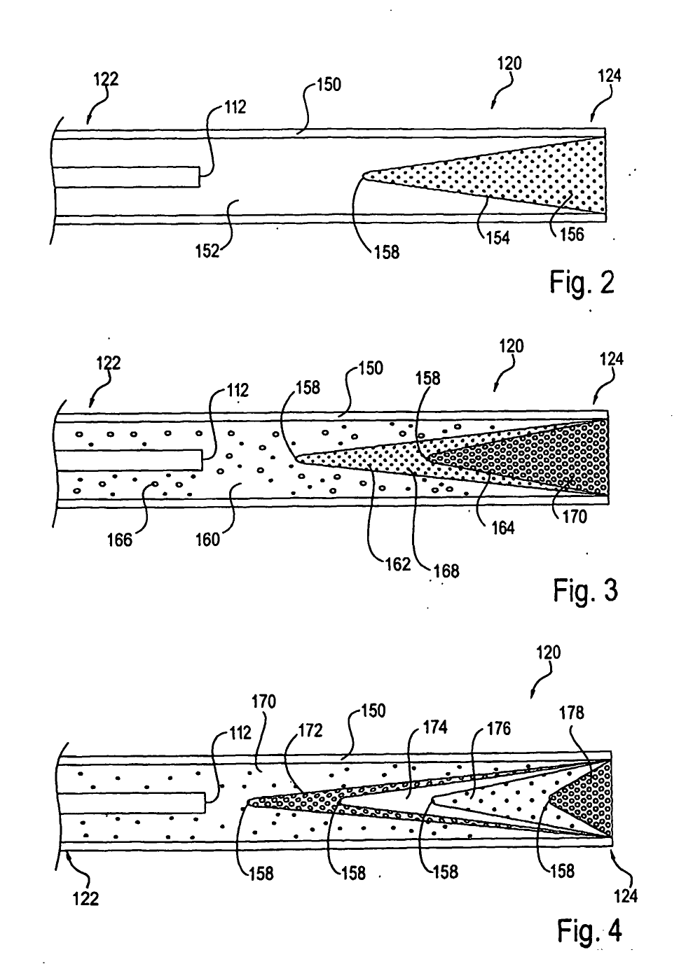

FIGS. 2-4 provide side views of various embodiments of the diffuser tip 120. Referring to FIG. 2, the diffuser tip 120 is shown including its proximal end 122 and distal end 124. The light-transmitting end portion 112 of the light guide 102 is shown disposed within the proximal end 122. Typically, the light guide comprises an optical fiber having a buffer layer which is stripped back to create the light-transmitting end portion. External tubing 150 provides a housing for the diffuser tip 120 which contains one or more light scattering mediums. In this embodiment, two regions are shown, a first region 152 comprising a transparent material having no scattering properties and a second region 154 comprising a light scattering medium. Examples of light scattering mediums include titanium dioxide, barium sulfate, powder quartz (SiO₂), aluminum oxide (Al₂O₃), polystyrene microspheres, silica microspheres, powdered diamond, zirconium oxide, ditantalum pentoxide, calcium hydroxyapatite, and a combination of any of these to name a few. In addition, the light scattering mediums may include particles which provide optical properties other than scattering. Such optical properties may allow the region to be used for detectors, sensors or MRI-guided placement of the diffuser tip, in addition to light therapy treatment. This may reduce the need for fluorscopy in placement of the diffuser tip. Examples of such particles include light absorbing particles, fluorescent particles, or magnetic resonance imaging (MRI)-detectable particles, such as Motexafin Gadolinium. In each case, the light scattering medium comprises a base material within which is embedded scattering particles 156. Generally, materials having higher concentrations of scattering particles 156 provide higher scattering power. In addition, certain types and sizes of scattering particles 156 may provide higher scattering power when in the same concentration. In this embodiment, the second region 154 has a conical shape wherein its apex 158 is directed toward the light transmitting end portion 112.

Referring to FIGS. 3-4, embodiments of the diffuser tip 120 may include more than two regions, each region having different concentrations of light scattering particles ranging from no particles to approximately 5-15% particles. It may be appreciated that the quantity of particles used depends on the type of the particles, the type of the base material and the relative size of the particles to the delivered wavelength of light. FIG. 3 illustrates an embodiment having a first region 160, a second region 162 and a third region 164, each region comprised of light scattering mediums having a different concentration or type of light scattering particles 156. Differences in concentration or type are illustrated by differences in particle density and size. As illustrated, the first region 160 has the lowest concentration of scattering particles 166, the second region 162 has a higher concentration of scattering particles 168 and the third region 164 has a similar concentration but different type of scattering particles 170 relative to the second region 162. In this example, the scattering power of the diffusive tip 120 increases from the proximal end 122 to the distal end 124. In addition, the second region 162 and third region 164 are conical in shape, each having their respective apex 158 directed toward the light-transmitting end portion 112.

FIG. 4 illustrates an embodiment having a first region 170, a second region 172, a third region 174, a fourth region 176 and a fifth region 178. Again, each region is comprised of light scattering mediums having a different concentration or type of light scattering particles 156. And, differences in concentration or type are illustrated by differences in particle density and size. As shown, two regions, such as the first region 170 and the fourth region 176 may have the same type and/or concentration of scattering particles if they are separated by another region, such as the second region 172. In addition, two regions containing scattering particles, such as the second region 172 and the fourth region 176, may be separated by a region having no scattering particles, such as the third region 174. Thus, any combination of regions may be used to create a diffuser tip 120 having unique scattering properties and hence illumination profile. In addition, in the embodiment, the second region 172, third region 174, fourth region 176 and fifth region 178 are all shown as having conical shapes with their respective apex facing the light-transmitting end portion 112. Although this orientation of the conical regions is preferred, it is not necessary and other embodiments having different orientations will be discussed in later sections.

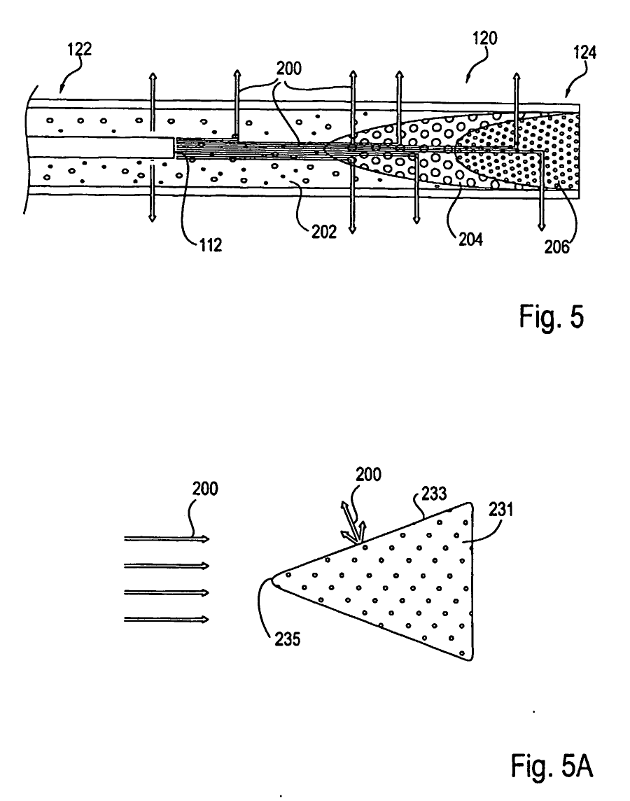

FIG. 5 illustrates the diffusion of light rays 200 (illustrated as arrows) which are transmitted from a light source, delivered from the light guide and diffused through the diffuser tip 120. A majority of the light rays 200 are shown exiting the light transmitting end portion 112. Rays 200 which travel axially along the diffuser tip 120 are redirected by interference with scattering particles, as shown. The light generally exits within a cone which half angle is determined by the numerical aperature of the fiber. Although scattered rays are illustrated as directed at a right angle to the axis, it may be appreciated that scattered rays are directed in substantially all directions. This embodiment of the diffuser tip 120 includes a first region 202 comprising a first medium having a first concentration of scattering particles, a second region 204 comprising a second medium having a second concentration of scattering particles, and a third region 206 comprising a third medium having a third concentration of scattering particles. As shown, rays 200 entering the first region 202 are scattered by the scattering particles. In this embodiment, rays 200 continuing to the second region 204 are scattered to a higher degree due to a higher scattering power of the second medium. Since less rays 200 enter the second region 204 compared with the first region 202, the scattered output may be approximately the same from the two regions. In addition, the conical shape of the second region 204 provides both a gradual transition between the scattering powers of the two regions and an interface which scatters the rays 200 in a desirable fashion. Referring to FIG. 5A, a light ray 200 entering a conical region 231 having scattering properties will be scattered by the region 231 at its surface 233 (interface) with a Lambertian (cosine) angular distribution. Consequently, a majority of the light rays 200 are scattered radially by the conical region 231 and minimal rays 200 are backscattered toward the tip 235 of the conical region 231 and therefore the fiber end. Thus, the conical shape results in a highly efficiency diffuser tip.

Referring back to FIG. 5, rays 200 continuing to the third region 206 are scattered to a higher degree due to a higher scattering power of the third medium. Since less rays 200 enter the third region 204 compared with the first region 202 and second region 204, the scattered output may be approximately the same all three regions. And, the conical shape of the third region 206 again provides both a gradual transition between the scattering powers of the two regions and an interface which scatters the rays 200 in a desirable fashion. Thus, the regions may be shaped, arranged and comprised of specific mediums which will effectively scatter substantially all light rays 200 entering the diffuser tip 120 before the rays 200 reach the distal end 124. Thus, all light transmitted to the most distally positioned region is substantially diffused outwardly. In this case, there would be no need to fix a reflective mirror at the distal end 124. The elimination of the mirror provides a number of benefits both in manufacture of the diffuser tip 120 and in use of the apparatus 100. In particular, such elimination of a need for a reflective mirror allows the diffuser tip 120 to be easily manufactured having a maximum outside diameter in the range of 100 μm to 2000 μm, preferably 250 μm to 1200 μm, more preferably 250 μm to 500 μm, including 0.014 inches (350 μm) which would allow introduction of the tip 120 into human coronary arteries or 0.018 inches (450 μm), or more preferably 800 μm to 1200 μm.

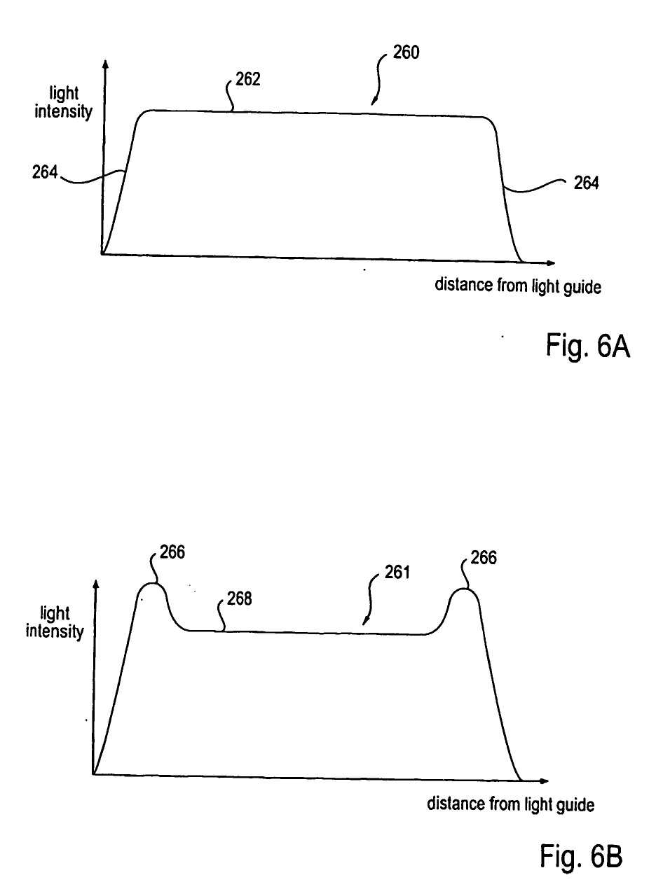

FIG. 6A illustrates a graphical representation of a scattered light illumination profile 260 or pattern of illumination from a diffuser tip 120 such as from the embodiment shown in FIG. 5. The profile 260 illustrates the light intensity of the scattered light rays relative to the distance from the light guide measured axially along the diffuser tip 120. As shown, the diffuser tip 120 provides a substantially uniform illumination profile 260, within approximately +/−20% uniformity. Light exiting the diffuser tip 120 has essentially the same intensity from near the proximal end 122 to near the distal end 124 of the diffuser tip 120. This is illustrated by the plateau 262 between the side edges 264. Alternatively, the regions may be shaped, arranged and comprised of specific mediums which will provide different illumination profiles. For example, as shown in FIG. 6B, the light intensity may be increased near the proximal and distal ends 122, 124, as illustrated by peaks 266, relative to a plateau 268 of lesser intensity therebetween. This profile 261 may compensate for effects near the ends 122, 124 of the diffuser tip 120 which would otherwise provide diminished light intensity. Thus, any desired illumination profile may be achieved by altering the shape, size, arrangement, orientation, choice of scattering medium, concentration of scattering particles and other variables related to the regions within the diffuser tip.

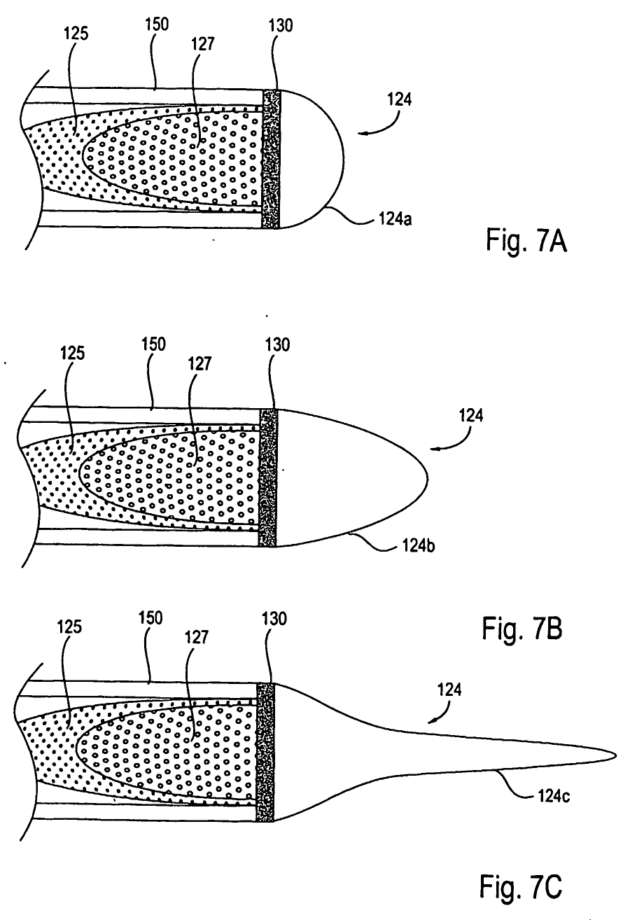

Example embodiments of the distal end 124 of the diffuser tip 120 are illustrated in FIGS. 7A-7C. The distal end 124 may have a shape adapted for use in treating specific target locations. Typically, such a shape is adapted for use in treating body lumens or in reaching target locations which are accessible by lumen shaped pathways. For such useage, a rounded or curved shaped distal end 124a may be desired, as shown in FIG. 7A. Or, a short, smooth, tapered distal end 124b may be desired, as shown in FIG. 7B. And in some cases, an extended, floppy distal end 124c or narrow elongated portion which is floppy may be desired, as shown in FIG. 7C, comprised of a flexible material to provide a floppy feel such as provided by a guidewire. In preferred embodiments, the floppy distal end 124c has a length of at least 10 mm. In each of these example embodiments, the distal end 124 is shaped to reduce any possible trauma to the body lumen or tissue of the target location upon delivery of the diffusion apparatus 100. Also, each of FIGS. 7A-7C illustrate the distal end 124 adjacent to a radiopaque marker 130 which is positioned near the end of the external tubing 150 having a first region 127 and second region 125 of scattering material therein. It may be appreciated that such features of the apparatus 100 are illustrated for the purposes of example only and any shaped distal end 124 may be present with or without a radiopaque marker 130 or various regions of scattering materials, etc. It may also be appreciated that embodiments illustrated throughout may have any shaped distal end and are not limited to the shaped illustrated, often a flat end.

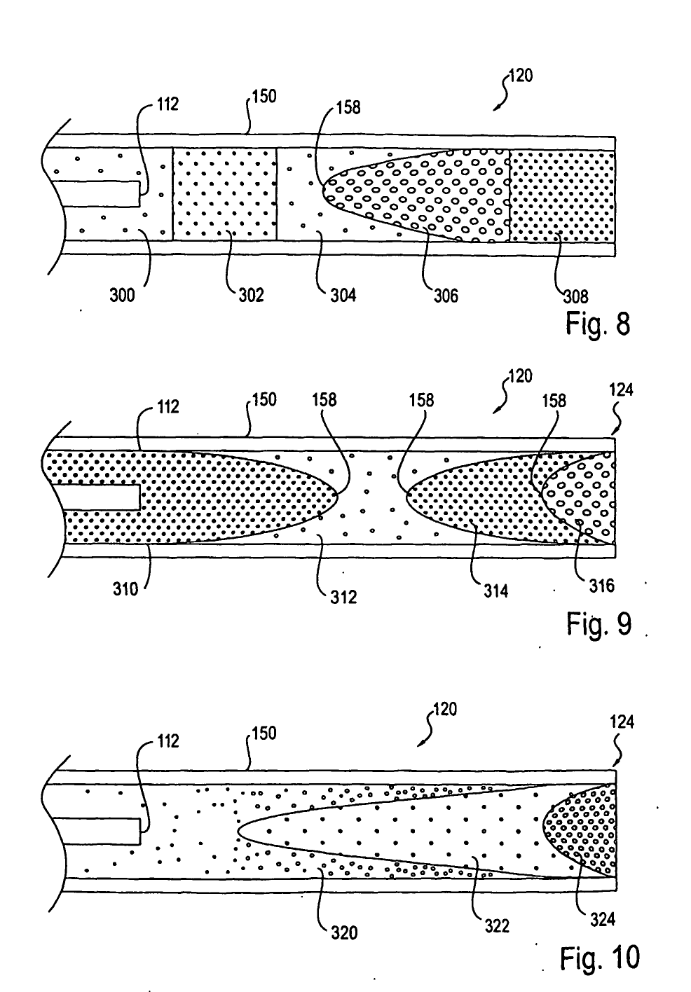

Additional embodiments of the diffuser tip 120 are illustrated in FIGS. 8-10. Until this point, embodiments have been shown with all regions, aside from the region adjacent the light transmitting end portion 112, as conical in shape having an orientation in which the apex 158 is directed toward the end portion 112. However, such shape, orientation and arrangement are not necessary for all regions. In the embodiment shown in FIG. 8, the diffuser tip 120 is comprised of a first region 300, a second region 302, a third region 304, a fourth region 306 and a fifth region 308. Each region may be comprised of different light scattering mediums, each having a different concentration and/or type of light scattering particles, no light scattering particles or the same concentration or type but separated by a region of a different concentration or type of particles. As shown, regions, such as the second region 302 and the fifth region 308, may be square or rectangular in shape while regions, such as the fourth region 306 may be conical in shape. Similarly, as shown in FIG. 9, which has a first region 310, a second region 312, a third region 314 and a fourth region 316, a conical region may be oriented so its apex 158 is directed toward the distal end 124, as illustrated by the first region 310. This may be combined with conical regions which are oriented so their apexes 158 are directed toward the end portion 112, as illustrated by the third and fourth regions 314, 316.

Referring to FIG. 10, any region may be comprised of a light scattering medium having a concentration of light scattering particles which is not uniform. For example, in this embodiment, having a first region 320, a second region 322, and a third region 324, the first region 320 comprises a light scattering medium having light scattering particles which increase in concentration toward the distal end 124 of the diffuser tip 120. This may be combined with regions, such as the second region 322 and the third region 324 which have uniform concentrations of scattering particles. In addition, in all embodiments of the diffuser tip 120, the external tubing 150 may also have scattering properties.

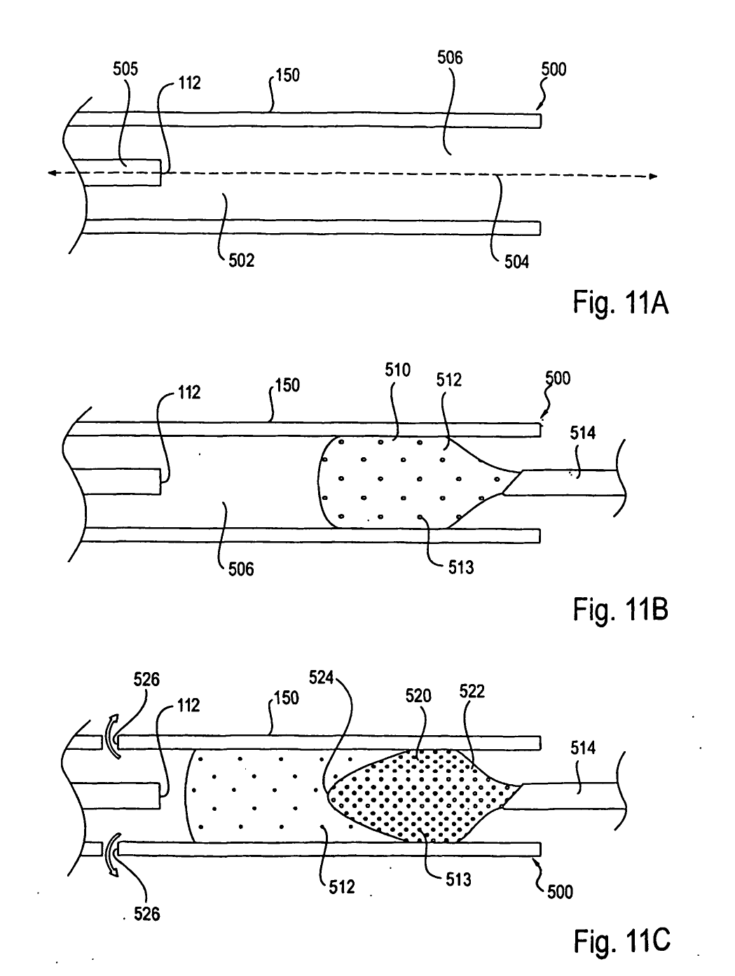

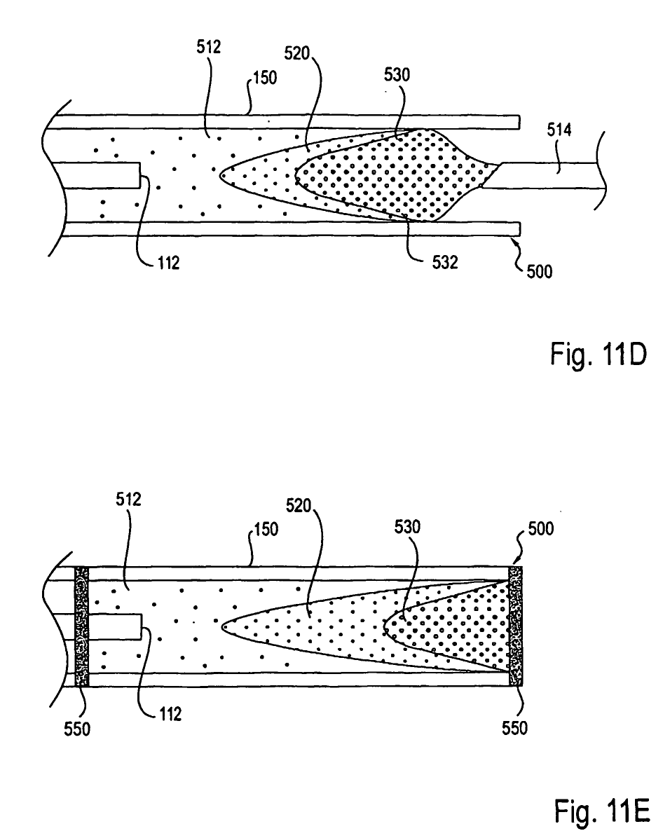

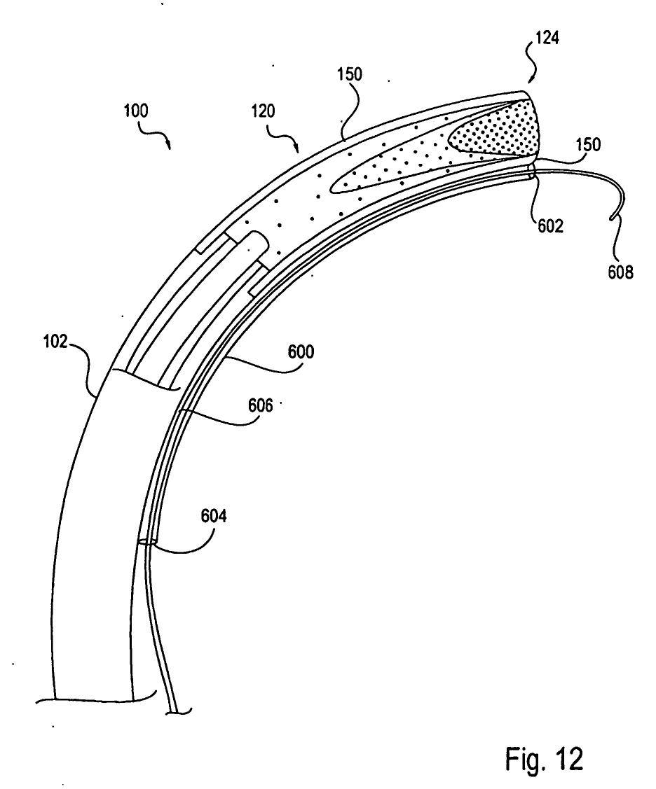

FIGS. 11A-11E illustrate how the present invention may be processed in manufacturing. Referring to FIG. 11A, the process involves a step of providing a segment of external tubing 150 having a proximal end (not shown), a distal end 500 and a lumen therethrough 502 having a center axis 504. The tubing 150 is typically in the range of 10 to 150 mm in length and has an outer diameter in the range of 100 to 2000 microns. For applicability to specific procedures, the tubing may have an outer diameter within one of three general ranges, 250 μm to 500 μm, 400 μm to 800 μm, and 800 μm to 1200 μm. An optical light guide 505 having an light transmitting end portion 112 is disposed within the tubing 150 so that there is a luminal space 506 between the end portion 112 and the distal end 500. The distance between the end portion 112 and the distal end is typically in the range of approximately 5 to 150 mm. The light guide 505 may be a standard optical fiber suitable for transmitting ultraviolet, visible, and near infrared light. The optical fiber is stripped of its buffer to expose at one end thereof a length of cladding and core which includes the light transmitting end portion 112. The diameter of the cladding and core together is typically in the range of 50-1900 microns. FIG. 11B illustrates a step of creating a first region 510 by injecting a first medium 512 into the luminal space 506 between the end portion 112 and the distal end 500. The first medium 512 may comprise a transparent medium having substantially optically clear properties, it may include scattering particles 513 (as shown) providing a desired light scattering power, or it may provide scattering properties by other means. Such mediums may include titanium dioxide, barium sulfate, powder quartz (SiO₂), aluminum oxide (Al₂O₃), polystyrene microspheres, silica microspheres, powdered diamond, zirconium oxide, ditantalum pentoxide, calcium hydroxyapatite, and a combination of any of these to name a few. The medium 512 may be injected through an injection tube 514 or any other means suitable for injecting such a medium. FIG. 11C illustrates a step of creating a second region 520 by injecting a second medium 522 into the distal end 500 of the external tubing 150 wherein the second region 520 has a conical shape. The second medium 522 has optical properties which differ from the first medium 512. For example, the second medium 522 may include optical particles 513 having a concentration which differs from that in the first medium 512. As the second medium 522 is injected into the tubing 150, the second medium 522 essentially pushes the first medium 512 through the tubing 150 toward the end portion 112. Fluid flowing through and filling a horizontal tube are acted on by a number of forces including inertia and friction. When a fluid flows into a tube, such as by injection, a boundary layer starts at the entrance and grows continuously until it cross-sectionally fills the tube. The boundary layer is the region in which the velocity of the fluid varies from 0 to V (a maximum velocity). Thus, the velocity is close to zero near the walls of the tubing 150 and reaches a maximum near the central axis 504. Since the second medium 522 is traveling at a higher velocity near the central axis 504 of the lumen 502, the second region 520 forms a conical shape wherein the apex 524 is directed toward the end portion 112. Displaced first medium 512 is pushed toward the end portion 112. As shown, venting ports 526 through the external tubing 150 may be located near the end portion 112 so that air and/or excess medium may escape through the ports 526 as illustrated by arrows.

FIG. 11D illustrates a step of creating a third region 530 by injecting a third medium 532 into the distal end 500 of the external tubing 150 wherein the third region 530 has a conical shape. The third medium 532 has optical properties which differ from the second medium 522 but may be the same as the first medium 512. Similar to the step of injecting the second medium 522, injection of the third medium 532 into the tubing 150 essentially pushes the second medium 522 and first medium 512 through the tubing 150 toward the end portion 112. Since the third medium 532 is traveling at a higher velocity near the central axis 504 of the lumen 502, the third region 530 forms a conical shape wherein the apex 524 is directed toward the end portion 112. It may be appreciated that the length and shape of the cones may be controlled by the method of injection, including speed of injection, angle and position of the injection tube 514 and a variety of other variables. In addition, regions may be non-conical shaped by using other methods of injection. In this case, a diffuser tip 120 as shown in FIG. 8 may be produced wherein a non-conical region, the third region 304, is followed by a conical region, the fourth region 306, which is in turn followed by a non-conical region, the fifth region 308. Further, conical regions, such as the first region 310 in FIG. 9, wherein the apex 158 is not directed toward the end portion 112 may be produced by injecting material through the tubing 150 wall toward the distal end 124 or by producing the diffuser tip 120 itself and then connecting the diffuser tip 120 to the light guide 102.

In any case, the above process steps may be repeated to create any number of regions in the diffuser tip 120. In the end, lumen 502 of the external tubing 150 will be filled with material. An example of such a diffuser tip 120 is illustrated in FIG. 11E. In addition, radiopaque marker bands 550 have been added to aid in visualization under fluoroscopic conditions. Such bands 550 may be applied to the outer surface of the external tubing 150 or may be located within the tubing 150. Alternatively, other radiopaque markings may be used, such as paint, or an injected medium may be comprised of a material having radiopacity properties or a material having a high concentration of scattering particles with radiopacity properties, such as Barium sulfate.

Referring to FIG. 12, the light transmission and diffusion apparatus 100 may optionally include a guidewire tubing 600 having a distal end 602, a proximal end 604, and a lumen 606 therethrough through which a guidewire 608 may pass. Typically, the guidewire lumen 606 is disposed along an axis parallel to the central axis, such as when the guidewire tubing 600 is disposed along the outside of the external tubing 150. The guidewire lumen 606 and may extend from the distal end 124 of the diffuser tip 120 to the proximal end 104 (not shown) of the light guide 102 or to any location therebetween. Often, the distal end 602 of the guidewire lumen 606 is aligned with the distal end 124 of the diffuser tip 120 and the proximal end 604 of the guidewire lumen 606 is located in the range of 20 to 30 cm from the distal end 602. Such an arrangement provides a “monorail” system which provides a number of benefits during treatment of a target site. In particular, the monorail system allows the guidewire 608 to be positioned within the guidewire tubing 600 during delivery of light therapy to the target site. In the area of the diffuser tip 120, the guidewire tubing 600 is comprised of a transparent material that allows passage of visible light, particularly 730 nm light, so that the guidewire tubing 600 will not interfere with the delivery of light to the target region. Depending on the position and material of the guidewire 608, the guidewire 608 may possibly obstruct light in this area but any possible effects on the therapeutic index would be within acceptable limits. Guidewire tubing 600 along any other portion of the apparatus 100 may be comprised of the same transparent material or it may be opaque, colored or have other properties. In addition, the guidewire tubing 600 may be a separate tube which is affixed or adhered to the outside of the external tubing 150, which may extend from the distal end 124 to the proximal end 104, or the guidewire lumen 606 may be formed as an extruded lumen within the walls of the apparatus 100.

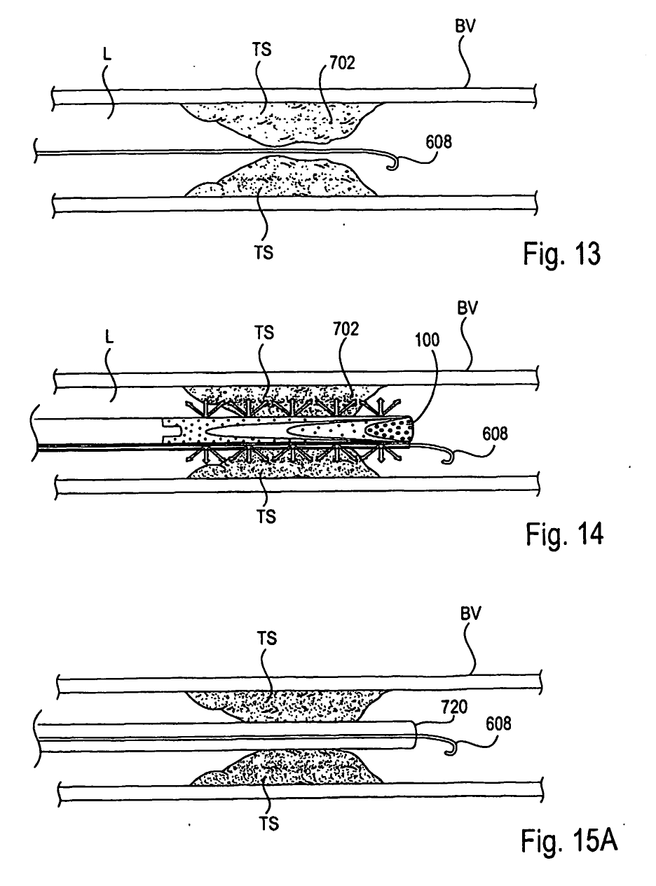

FIGS. 13, 14, 15A-15B and 16A-16B illustrate methods of using the present invention. In particular, such embodiments illustrate methods of performing photodynamic therapy at a target site within a body lumen. It may be appreciated that the present invention may also be used interstitially or in non-cylindrical body cavities and may be used for purposes other than photodynamic therapy. FIG. 13 illustrates a cross-sectional view of a target site TS within a body lumen L. In this case, the target site TS is a stenosis of atheromatous material within a blood vessel BV. As shown, a photosensitive compound 702 has been introduced into the target site TS to be activated by delivered light. The target site TS may be accessed by any means appropriate and a guidewire 608 may be positioned through the target site TS as shown. When accessing a target site TS in a blood vessel BV, a percutaneous approach is often used such that a location of the vasculature remote from the target site TS is accessed through the skin, such as using needle access as in the Seldinger technique or by performing a surgical cut down procedure or minimally invasive procedure. In any case, the ability to percutaneously access the remote vasculature and position a guidewire therein is well-known and described in the patent and medical literature.

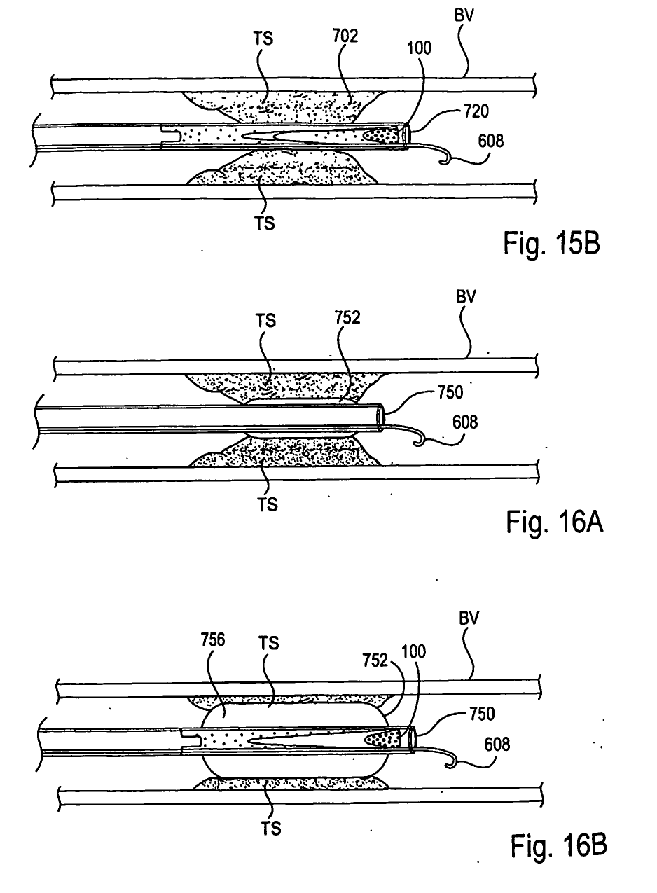

Referring to FIG. 14, the distal end 124 of the diffuser tip 120 of the light transmission and diffusion apparatus 100 is introduced to the target site TS. In this case, the apparatus 100 is tracked over the guidewire 608 and positioned such that the diffuser tip 120 is positioned within the target site TS. The apparatus 100 is then coupled to light radiation, such as from a light source 110, so that light received by the diffuser tip 120 is delivered to the target site TS, as illustrated by arrows. Such light delivery activates the photosensitive compound 702 causing therapeutic effects. Alternatively, as shown in FIG. 15A, a catheter 720, such as a Transit™ catheter, may be positioned within the target site TS by tracking over the guidewire 608. Typically the catheter 720 will have a single lumen, be compatible with 0.018″ guidewires and have a floppy distal segment. The guidewire 608 is then removed and the apparatus 100 may then be introduced through the catheter 720 so that the diffuser tip 120 is also positioned within the target TS, as shown in FIG. 15B. The apparatus 100 may then deliver light to the target site TS wherein the light travels radially through the walls of the catheter 720. In this case, the catheter 720 is comprised of a transparent material, to allow transmission of light, or a material having optical scattering properties. Alternatively, the catheter 720 may be retracted while the apparatus 100 remains in place. In this case, light received by the diffuser tip 120 is delivered to the target site TS as illustrated in FIG. 14.

Referring to FIG. 16A, a balloon catheter 750 having a balloon 752 mounted on its distal end 754 may be positioned within the target site TS by tracking over the guidewire 608. In this example, the balloon 752 is positioned within the target site TS as desired to perform an angioplasty procedure. As shown in FIG. 16B, the balloon 752 is then inflated with inflation fluid 756 thereby opening up the stenosis by compressing the atheromatous material against the walls of the blood vessel BV. While the balloon 752 is inflated, the guidewire 608 may or may not be removed and the apparatus 100 may be introduced through the balloon catheter 750 so that the diffuser tip 120 is also positioned within the target TS, as shown in FIG. 16B. The apparatus 100 may then deliver light to the target site TS wherein the light travels radially through the balloon 752. In this case, the materials comprising the balloon catheter 750, balloon 752 and the inflation fluid 756 are transparent, to allow transmission of light, or have optical scattering properties. It may be appreciated that some materials may be transparent while others have optical scattering properties. Alternatively, the balloon 752 may be deflated and the balloon catheter 750 may be retracted while the apparatus 100 remains in place. In this case, light received by the diffuser tip 120 is delivered to the target site TS as illustrated in FIG. 14.

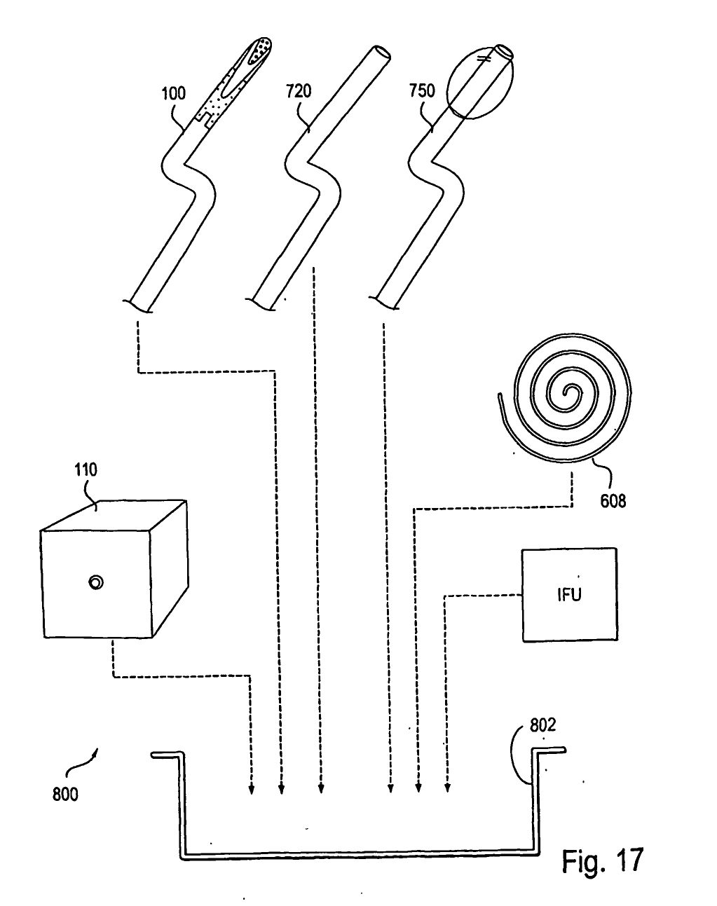

Referring now to FIG. 17, kits 800 according to the present invention comprise at least a light transmission and diffusion apparatus 100 and instructions for use IFU. Optionally, the kits 800 may further include any of the other components described above, such as a catheter 720, a balloon catheter 750, a guidewire 608, and a light source 110. The instructions for use IFU will set forth any of the methods as described above, and all kit components will usually be packaged together in a pouch 802 or other conventional medical device packaging. Usually, those kit components, such as the apparatus 100, which will be used in performing the procedure on the patient will be sterilized and maintained within the kit. Optionally, separate pouches, bags, trays or other packaging may be provided within a larger package, where the smaller packs may be opened separately to separately maintain the components in a sterile fashion.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended claims.

1-90. (canceled)

91. A light transmission and diffusion apparatus comprising:
a light guide having a proximal end and distal end, the proximal end adapted for coupling to a light source and the distal end having a light-transmitting end portion; and
a diffuser tip having a proximal end enclosing said end portion and a distal end, the diffuser tip comprising at least a first region and a second region, the second region comprising a second light scattering medium having a second concentration of scattering particles and wherein the second region has a conical shape and is proximal to the distal end of the diffuser tip.

92. An apparatus as in claim 91, wherein the first region comprises a first light scattering medium having a first concentration of scattering particles.

93. An apparatus as in claims 91 or 92, wherein the first and second regions are positioned and their light scattering mediums and concentrations of scattering particles are chosen such that the diffuser tip produces a substantially uniform pattern of illumination during light transmission.

94. An apparatus as in claim 93, wherein the substantially uniform pattern of illumination is within approximately +/−20% uniformity.

95. An apparatus as in claim 93, wherein the substantially uniform pattern of illumination comprises light of essentially the same intensity from near the proximal end if the diffuser tip to near the distal end of the diffuser tip.

96. An apparatus as in claims 91 or 92, wherein the regions are positioned and their light scattering mediums and concentration of scattering particles are chosen such that the diffuser tip produces a pattern of illumination during light transmission which has an intensity at its proximal and distal ends which is greater than the intensity therebetween.

97. An apparatus as in claim 92, wherein the second region is distal to the first region.

98. An apparatus as in claim 97, wherein the second concentration of scattering particles is greater than the first concentration of scattering particles.

99. An apparatus as in claim 98, wherein the second region is oriented so its apex is directed toward the light-transmitting end portion.

100. An apparatus as in claim 92, wherein the diffuser tip further comprises a third region comprising a third light scattering medium having a third concentration of scattering particles and wherein the third region has a conical shape.

101. An apparatus as in claim 100, wherein the third region is oriented so its apex is directed toward the light-transmitting end portion and is distal to and nested within the second portion.

102. An apparatus as in claim 101, wherein the third concentration of scattering particles is greater than the second concentration of scattering particles.

103. An apparatus as in claim 100, wherein the diffuser tip further comprises a fourth region comprising a fourth light scattering medium having a fourth concentration of scattering particles and wherein the fourth region has a conical shape, wherein the fourth region is oriented so its apex is directed toward the light-transmitting end portion and is distal to and nested within the third portion and wherein the fourth concentration of scattering particles is greater than the third concentration of scattering particles.

104. An apparatus as in claim 92, 100 or 103, wherein each region comprises a different light scattering medium.

105. An apparatus as in claim 92, 100 or 103, wherein each region comprises a different concentration of scattering particles.

106. An apparatus as in claim 92, 100 or 103, wherein each region comprises scattering particles having a different size.

107. An apparatus as in claim 92, 100 or 103, wherein each region comprises scattering particles having a different refractive index.

108. An apparatus as in claim 92, 100 or 103, wherein each region comprises scattering particles having different absorption properties.

109. An apparatus as in claim 92, 100 or 103, wherein each region further comprises particles which are light absorbing, fluorescent or magnetic resonance imaging detectable.

110. An apparatus as in claim 91, wherein the light scattering medium of the most distally positioned region provides radiopacity under fluoroscopy.

111. An apparatus as in claim 110, wherein the light scattering medium of the most distally positioned region comprises barium sulfate, ditantalum pentoxide or calcium hydroxyapatite.

112. An apparatus as in claim 91, wherein the regions are positioned and their light scattering mediums and concentration of scattering particles are chosen such that all light transmitted to the most distally positioned region is substantially diffused radially outwardly.

113. An apparatus as in claim 112, wherein the light scattering mediums comprise titanium dioxide, barium sulfate, powder quartz, aluminum oxide, polystyrene microspheres, silica microspheres, powdered diamond, zirconium oxide, ditantalum pentoxide, calcium hydroxyapatite, or a combination of any of these.

114. An apparatus as in claim 91, wherein the diffuser tip has a maximum outside diameter in the range of about 150 μm to 1200 μm.

115. An apparatus as in claim 114, wherein the diffuser tip has a maximum outside diameter in the range of about 250 μm to 1200 μm.

116. An apparatus as in claim 114, wherein the diffuser tip has a maximum outside diameter of approximately 250 μm to 500 μm.

117. An apparatus as in claim 114, wherein the diffuser tip has a maximum outside diameter of approximately 800 μm to 1200 μm.

118. An apparatus as in claim 91, wherein the diffuser tip further comprises an external layer comprising light scattering material.

119. An apparatus as in claim 91, wherein the distal end has a rounded or short tapered shape.

120. An apparatus as in claim 91, wherein the distal end terminates in a narrow elongated portion which is floppy.

121. An apparatus as in claim 91, further comprising a guidewire lumen.

122. An apparatus as in claim 121, wherein the guidewire lumen is disposed along an axis parallel to and offset from a central axis.

123. An apparatus as in claim 91, wherein the diffuser tip is adapted to be insertable within a lumen in a catheter.

124. An apparatus as in claim 123, wherein the catheter has a balloon mounted thereon and the diffuser tip is insertable to a position where the tip is surrounded by the balloon.
